(19) United States
(12) Patent Application Publication
(10) Pub. No.: US 2026/0250382 A1
(43) Pub. Date: Aug. 27, 2026

(54) METHODS OF TREATMENT OF GLOMERULONEPHROPATHY

(71) Applicant: Climb Bio Operating Inc., Wellesley, MA (US)

(72) Inventors: Nishi Rampal, Wellesley, MA (US); Jan Hillson, Wellesley, MA (US); Aoife Brennan, Wellesley, MA (US); William Bonificio, Wellesley, MA (US); Stephen Thomas, Wellesley, MA (US)

(21) Appl. No.: 19/355,188

(22) Filed: Oct. 10, 2025

Related U.S. Application Data
(60) Provisional application No. 63/706,854, filed on Oct. 14, 2024.

(51) Int. Cl. C07K 16/28; A61P 13/12

(52) U.S. Cl. CPC C07K 16/2803; A61P 13/12

(57) ABSTRACT

The invention provides novel methods for treatment of patients with minimal change disease (MCD) or focal segmental glomerulosclerosis (FSGS) by administration of compositions comprising a therapeutic agent. According to the invention, the therapeutic agent for the treatment of MCD and/or FSGS may be an anti-CD19 antibody.

### I. FIELD OF THE INVENTION

The invention is directed to novel methods for treatment of patients with glomerulonephropathy by administration of a composition comprising a therapeutically effective amount of an anti-CD19 antibody.

### II. SEQUENCE LISTING

The present application is being filed along with a Sequence Listing XML in electronic format. The Sequence Listing XML is provided as a file named CLIMB-007-01US-Seq-Listing.xml, created on Oct. 8, 2025, which is 55,758 bytes in size. The Sequence Listing XML file and the information in the electronic format of the Sequence Listing XML file is incorporated herein by reference in its entirety.

### III. BACKGROUND

The functioning of kidneys depends on the glomeruli—the mass of tiny, semipermeable tubes deep in the kidneys through which the blood passes, filtering out water and soluble wastes, which then travel to the bladder and out of the body through the ureters and urethra. These tubes are subject to injury from several sources including viruses, bacteria, certain immune diseases such as lupus, and other conditions such as hypertension. Symptoms of such kidney problems include fatigue, nausea, hematuria (pink or dark-colored urine due to red blood cells), proteinuria (bubbly urine from excess protein), hypertension (high blood pressure) and edema (fluid retention) causing swelling of face, hands and feet.

Glomerulonephropathy is a disease that damages the glomeruli, or filters, in the kidneys. This damage can cause protein and red blood cells to leak into the urine, which can lead to kidney failure. Glomerulonephropathy may also result from other conditions including, minimal change disease (MCD), focal segmental glomerulosclerosis (FSGS), and lupus nephritis (LN).

### IV. SUMMARY OF THE INVENTION

The invention provides novel methods for treatment of glomerulonephropathy. The patients may be suffering from glomerulonephropathy associated with autoantibodies. The patients may have glomerulonephropathy due to other underlying pathology. For example, glomerulonephropathy may be due to MCD or FSGS.

There is an unmet need for improved therapies for steroid-dependent and frequently relapsing MCD and FSGS, and there are currently no Food and Drug Administration-approved treatments specifically indicated for primary MCD or primary FSGS. Patients with MCD and FSGS suffer frequent disease flares and complications from long-term corticosteroid exposure. The invention provides a novel therapeutic approach for reducing inflammation and injury to kidney through depletion of CD19+ cells through compositions comprising anti-CD19 antibodies. The invention provides methods of treatment of glomerulonephropathy associated with MCD and FSGS. In certain beneficial aspects, the methods provided herein reduce proteinuria and maintain remission in MCD/FSGS patients.

A significant challenge in the treatment of patients with MCD is that a significant number of patients have become steroid dependent, which is the current standard of care for the treatment of MCD patients. These patients are unable to taper steroids without a relapse. In addition, this is a frequently relapsing disease.

In certain aspects, the invention provides a method of treating a subject having glomerulonephropathy, wherein the method comprises administration of a composition comprising a therapeutically effective amount of an anti-CD19 antibody. In certain embodiments, glomerulonephropathy is associated with the generation of autoantibodies. In certain embodiments, glomerulonephropathy is selected from the group consisting of minimal change disease (MCD), focal segmental glomerulosclerosis (FSGS), and lupus nephritis (LN). In certain embodiments, the invention provides that immunomodulation by anti-CD19 antibody and reduction in number of CD19+ B-cells.

In certain embodiments, the patients are corticosteroid-dependent or frequently relapsing. In certain embodiments, the subject has previously received corticosteroid therapy and/or any additional therapy for treatment of nephrotic syndromes. In certain embodiments, the patients are responsive to corticosteroids. Accordingly, in certain embodiments, the patients receiving the anti-CD19 therapy are responsive to therapy with corticosteroids or steroids.

In certain embodiments, the additional therapies are selected from the group consisting of calcineurin inhibitors, mycophenolic acid analogues, azathioprine, and cyclophosphamide. In certain embodiments, the subject has received the corticosteroid therapy for at least 8 weeks before start of administration of the composition comprising a therapeutically effective amount of anti-CD19 antibody. In certain embodiments, the corticosteroid therapy is administration of prednisone. In certain embodiments, the prednisone is administered at a dose higher than 20 mg/day, the subject is tapered off to a dose of 20 mg/day of prednisone. In certain embodiments, tapering is done from at least eight weeks to about 2 weeks before starting administration of the composition. In certain embodiments, prednisone is administered at a dose of less than about 20 mg/day for at least two weeks before starting administration of the composition.

In certain embodiments, the anti-CD19 antibody of the invention could be any anti-CD19 antibody provided herein. In certain embodiments, wherein the anti-CD19 antibody comprises: a heavy chain variable region at least 95% identical to SEQ ID NO: 17 and/or a light chain variable region at least 95% identical to SEQ ID NO: 29.

In certain embodiments, wherein the anti-CD19 antibody comprises: a heavy chain variable region comprising SEQ ID NO: 17 and/or a light chain variable region comprising SEQ ID NO: 29. In certain preferred embodiments, the anti-CD19 antibody is budoprutug.

In certain embodiments, the composition comprising a therapeutically effective amount of anti-CD19 antibody is administered parenterally. In certain embodiments, the composition comprising a therapeutically effective amount of anti-CD19 antibody is administered via intravenous (IV) infusion. In certain preferred embodiments, the anti-CD19 antibody administered to the subject is budoprutug.

In certain embodiments, the composition comprising a therapeutic effective amount of anti-CD19 antibody is administered at days 1 and 15 at the start of the treatment. In certain embodiments, the composition comprises about 100 mg to about 500 mg of the anti-CD19 antibody. In certain embodiments, the composition comprises about 100 mg to about 400 mg of the anti-CD19 antibody. In certain embodiments, composition comprises about 100 mg to about 300 mg of the anti-CD19 antibody. In certain embodiments, composition comprises about 100 mg to about 200 mg of the anti-CD19 antibody. In certain preferred embodiments, composition comprises about 100 mg of the anti-CD19 antibody. In certain preferred embodiments, composition comprises about 200 mg of the anti-CD19 antibody.

In certain embodiments, the method further comprises administration of an additional cycle to the subject, wherein the additional cycle comprises administration of a first additional dose of the composition and a second additional dose of the composition. In certain embodiments, the concentration of B cells is measured to decide if the additional cycle is administered. A high B cell count indicates that the administration of the composition of the invention did not have the desired effect. In certain embodiments, the additional cycle is administered if B cell count in the subject is greater than 10 cells/μL.

In certain embodiments, the first additional dose of the additional cycle is administered between day 113 and day 155 after first administration of the composition to the subject. In certain embodiments, the first additional dose of the additional cycle is administered between day 113 and day 141 after first administration of the composition to the subject. In certain embodiments, the second additional dose is administered 14 days after administration of the first additional dose.

In certain embodiments, the first additional dose of the additional cycle is administered between day 169 and day 183 after first administration of the composition to the subject. In certain embodiments, the first additional dose and the second additional dose are administered if the subject has not previously received the first additional dose and the second additional dose between days 113 and 115 after first administration of the composition to the subject. In certain embodiments, the first additional dose and the second additional dose comprise about 100 mg of the anti-CD19 antibody. In certain embodiments, the first additional dose and the second additional dose comprise about 200 mg of the anti-CD19 antibody.

### V. BRIEF DESCRIPTION OF THE DRAWINGS

FIG. 1 provides the schematic of the design of Phase II trials.

### VI. DETAILED DESCRIPTION

The following is a detailed definition of the terms used in the specification.

The term “shelf-stable pharmaceutical composition” as used herein means a pharmaceutical composition which is stable for at least the period which is required by regulatory agencies in connection with therapeutic proteins. Preferably, a shelf-stable pharmaceutical composition is stable for at least one year at 5° C. Stability includes chemical stability as well as physical stability.

The term “effective amount” as used herein means a dosage which is sufficient in order for the treatment of the patient to be effective compared with no treatment.

The term “composition” or “pharmaceutical composition” as used herein means a product comprising an active compound or a salt thereof together with pharmaceutical excipients such as buffer, preservative and tonicity modifier, said pharmaceutical composition being useful for treating, preventing or reducing the severity of a disease or disorder by administration of said pharmaceutical composition to a person. Thus, a pharmaceutical composition is also known in the art as a pharmaceutical formulation. It is to be understood that pH of a pharmaceutical composition which is to be reconstituted is the pH value which is measured on the reconstituted composition produced by reconstitution in the prescribed reconstitution liquid at room temperature.

The term “pharmaceutically acceptable” as used herein means suited for normal pharmaceutical applications, i.e. giving rise to no adverse events in patients etc.

The term “buffer” as used herein refers to a chemical compound in a pharmaceutical composition that reduces the tendency of pH of the composition to change over time as would otherwise occur due to chemical reactions. Buffers include chemicals such as sodium phosphate, TRIS, glycine and sodium citrate.

### Anti-CD19 Antibodies of the Invention:

In certain aspects, the invention provides anti-CD19 antibodies which feature an amino acid sequence defining a modified immunoglobulin heavy chain framework region comprising amino acid residues 1-30 of SEQ ID NO:22, wherein one or more of the amino acid residues at positions X5, X12, X19, X20, X23, and X24 are as follows: X5 is Q or E, X12 is V or K, X19 is R or K, X20 is L or V, X23 is K, E or D, or X24 is T or A. In certain embodiments, at least one of the amino acid residues at positions X5, X12, X19, X20, X23, or X24 is not the same amino acid residue as the amino acid at the corresponding position in the unmodified immunoglobulin heavy chain framework region as set forth in amino acid residues 1-30 of SEQ ID NO:13. In one embodiment, X23 is E or D.

In certain embodiments, the anti-CD19 antibodies feature an amino acid sequence defining a modified immunoglobulin heavy chain framework region comprising amino acid residues 1-14 of SEQ ID NO:23, wherein one or more of the amino acid residues at positions X3, X5, X7, and X8, are as follows: X3 is K or R, X5 is R, T, or A, X7 is G, D, or E, or X8 is Q or K. According to this aspect of the invention, at least one of the amino acid residues at positions X3, X5, X7, or X8 is not the same as the amino acid at the corresponding position in the unmodified immunoglobulin heavy chain framework region as set forth in amino acid residues 36-49 of SEQ ID NO:13. In one embodiment, X7 is E or D.

In certain embodiments, the anti-CD19 antibodies feature an amino acid sequence defining a modified immunoglobulin heavy chain framework region comprising amino acid residues 1-39 of SEQ ID NO:24, wherein one or more of the amino acid residues at positions X6, X10, X26, X29, and X34 are as follows: X6 is K, D, or E, X10 is K, E, or D, X26 is S, D, or E, X29 is S or A, or X34 is V or T. According to this aspect of the invention, at least one of the amino acid residues at positions X6, X10, X26, X29, or X34 is not the same as the amino acid at the corresponding position in the unmodified immunoglobulin heavy chain framework region as set forth in amino acid residues 60-98 of SEQ ID NO:13. In one embodiment, X10 is E or D.

In certain embodiments, the anti-CD19 antibodies feature an amino acid sequence defining a modified immunoglobulin light chain framework region comprising amino acid residues 1-23 of SEQ ID NO:32, wherein one or more of the amino acid residues at positions X1, X3, X7, X10, X11, and X19 are as follows: X1 is Q or D, X3 is V or A, X7 is S or E, X10 is I or T, X11 is M or L, or X19 is V or A. According to this aspect of the invention, at least one of the amino acid residues at positions X1, X3, X7, X10, X11, or X19 is not the same as the amino acid at the corresponding position in the unmodified immunoglobulin light chain framework region as set forth in amino acid residues 1-23 of SEQ ID NO:25. In one embodiment, X3 is A and X7 is E. In another embodiment, X1 is D, X10 is I, and X11 is L.

In certain embodiments, the invention features an amino acid sequence defining a modified immunoglobulin light chain complementarity determining region comprising amino acid residues 24-33 of SEQ ID NO:28.

In certain embodiments, the invention features an amino acid sequence defining a modified immunoglobulin light chain framework region comprising amino acid residues 56-87 of SEQ ID NO: 28.

According to another aspect, the invention features an antibody variable region comprising an amino acid sequence selected from the group consisting of SEQ ID NO: 14, SEQ ID NO: 15, SEQ ID NO: 16, SEQ ID NO: 17, SEQ ID NO: 20, SEQ ID NO: 21, SEQ ID NO: 26, SEQ ID NO: 27, SEQ ID NO: 28, SEQ ID NO: 29, SEQ ID NO: 30, or SEQ ID NO: 31, wherein the antibody variable region specifically binds to CD19.

In certain embodiments, the invention features a polypeptide at least 90% or at least 95% identical to a B4 antibody heavy chain variable region, the polypeptide comprising an amino acid substitution at one or more residues corresponding to Val12, Leu20, Lys23, Thr24, Lys38, Gly42, Gln43, Lys65, Lys69, Ser85, Ser88, or Val93. In one embodiment, the polypeptide comprises one or more of substitutions Gln5Glu, Val12Lys, Arg19Lys, Leu20Val, Lys23Glu, Lys23 Asp, Thr24Ala, Lys38Arg, Arg40Thr, Gly42Asp, Gly42Glu, Gln43Lys, Lys65Asp, Lys65Glu, Lys69Glu, Lys69Asp, Ser85Asp, Ser85Glu, Ser88Ala, or Val93Thr.

According to another aspect, the invention features a polypeptide at least 90% or at least 95% identical to a B4 antibody light chain variable region, the polypeptide comprising an amino acid substitution at one or more residues corresponding to Val3, Ser7, Ile10, Met11, Val19, Val29, Ser51, Leu53, Ala54, or Ser75. In one embodiment, the polypeptide comprises one or more of substitutions Gln1Asp, Val3Ala, Ser7Glu, Ile10Thr, Met11Leu, Val19Ala, Val29Ala, Ser51Asp, Leu53Thr, Ala54Asp, or Ser75Glu.

In certain aspects, the invention provides a process for preparation of anti-CD19 antibody, wherein the anti-CD19 antibody comprises variable domain comprising a light chain variable region that is at least 90% identical to SEQ ID NO: 25 and has an amino acid substitution at one or more residues corresponding to Ile10, Met11, Val19, Ser51, and Leu53; and the process comprises production of the anti-CD19 antibody in Chinese Hamster Ovary (CHO) cells.

In certain aspects, the invention provides a process for preparation of an anti-CD19 antibody, wherein: the anti-CD19 antibody comprises SEQ ID NO: 13 with an amino acid substitution at one or more residues corresponding to Gln5, Arg19, Leu20, Arg40, Gln43, Lys65, Ser85, Ser88, and Val93, and SEQ ID NO: 25 with an amino acid substitution at one or more residues corresponding to Ile10, Met11, Val19, Ser51, and Leu53; and the process comprises production of the anti-CD19 antibody in Chinese Hamster Ovary (CHO) cells.

In certain embodiments, the anti-CD19 antibody variable domain comprises a heavy chain variable region of SEQ ID NO: 17 and a light chain variable region of SEQ ID NO: 29. In certain embodiments, the anti-CD19 antibody is provided in U.S. Pat. No. 8,691,952, which is incorporated by reference in its entirety. In certain embodiments, the anti-CD19 antibody variable domain comprises a heavy chain variable region of SEQ ID NO: 17 and a light chain variable region of SEQ ID NO: 29 are provided in U.S. Pat. No. 8,691,952.

In certain embodiments, the heavy chain variable region of the anti-CD19 antibody has, compared to SEQ ID NO:13, one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, the heavy chain variable region of the anti-CD19 antibody is SEQ ID NO: 13 comprising an amino acid substitution at one or more residues corresponding to Gln5, Arg19, Leu20, Arg40, Gln43, Lys65, Ser85, Ser88, and Val93.

In certain embodiments, the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody is the amino acid sequence of SEQ ID NO: 17.

In certain embodiments, the anti-CD19 antibody variable domain of the anti-CD19 antibody further comprises a light chain variable region that is at least 90% identical to SEQ ID NO: 25 and has an amino acid substitution at one or more residues corresponding to Ile10, Met11, Val19, Ser51, and Leu53.

In certain embodiments, wherein the light chain variable region the anti-CD19 antibody has one or more amino acid substitutions selected from the group consisting of Ile10Thr, Met11Leu, Val19Ala, Ser51Asp, and Leu53Thr.

In certain embodiments, the light chain variable region is at least 95% identical to SEQ ID NO: 25.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is SEQ ID NO: 25 comprising an amino acid substitution at one or more residues corresponding to Ile10, Met11, Val19, Ser51, and Leu53.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is the amino acid sequence of SEQ ID NO: 29.

In certain embodiments, the heavy chain variable region comprises one or more of substitutions Gln5Glu, Arg19Lys, Leu20Val, Arg40Thr, Gln43Lys, Lys65Asp, Ser85Asp, Ser88Ala, and Val93Thr.

In certain embodiments, wherein the light chain variable region comprises one or more of substitutions Ile10Thr, Met11Leu, Val19Ala, Ser51Asp, and Leu53Thr.

In certain embodiments, wherein the anti-CD19 antibody comprises: a heavy chain variable region comprising SEQ ID NO: 17 and/or a light chain variable region comprising SEQ ID NO: 29. In certain preferred embodiments, the anti-CD19 antibody is budoprutug (also known as VB119).

In certain embodiments, the anti-CD19 antibody is VB119. In certain embodiments, VB119 is an anti-CD19, IgG1 monoclonal antibody. VB119 demonstrates antibody-dependent cell mediated toxicity (ADCC) with minimal to no complement dependent cytotoxicity (CDC). In certain embodiments, VB119 is an anti-CD19 antibody comprising a heavy chain variable region comprising SEQ ID NO: 17 and/or a light chain variable region comprising SEQ ID NO: 29.

In certain embodiments, the anti-CD19 antibody comprises one or more of the below listed subunits in the anti-CD19 antibody:

Subunit 1 (SEQ ID NO: 50):
QVQLEQPGAEVVKPGASVKVSCKTSGYTFTSNWMHWVKQTPGKGLEWIGEIDP
SDSYTNYNQKFDGKAKLTVDKSSSTAYMEVSDLTAEDSATYYCARGSNPYYYAMDYW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Subunit 2 (SEQ ID NO: 51):
QVQLEQPGAEVVKPGASVKVSCKTSGYTFTSNWMHWVKQTPGKGLEWIGEIDP
SDSYTNYNQKFDGKAKLTVDKSSSTAYMEVSDLTAEDSATYYCARGSNPYYYAMDYW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Subunit 3 (SEQ ID NO: 52):
QIVLTQSPATLSASPGEKATMTCSASSGVNYMHWYQQKPGTSPKRWIYDTDKTAS
GVPARFSGSGSGTSYSLTISSMEAEDAATYYCHQRGSYTFGGGTKLEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
Subunit 4 (SEQ ID NO: 53):
QIVLTQSPATLSASPGEKATMTCSASSGVNYMHWYQQKPGTSPKRWIYDTDKTAS
GVPARFSGSGSGTSYSLTISSMEAEDAATYYCHQRGSYTFGGGTKLEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC

In certain embodiments, the heavy chain sequence of the anti-CD19 antibody is provided below (SEQ ID NO: 54):

QVQLEQPGAEVVKPGASVKVSCKTSGYTFTSNWMHWVKQTPGKGLEWIGEIDP
SDSYTNYNQKFDGKAKLTVDKSSSTAYMEVSDLTAEDSATYYCARGSNPYYYAMDYW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK

In certain embodiments, the light chain sequence of the anti-CD19 antibody is provided below (SEQ ID NO: 55):

QIVLTQSPATLSASPGEKATMTCSASSGVNYMHWYQQKPGTSPKRWIYDTDKTAS
GVPARFSGSGSGTSYSLTISSMEAEDAATYYCHQRGSYTFGGGTKLEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC

In certain embodiments, the anti-CD19 antibody is budoprutug. In certain embodiments, budoprutug comprises a heavy chain and a light chain. In certain embodiments, budoprutug comprises heavy chain and light chain provided in SEQ ID NO: 54 and SEQ ID NO: 55 respectively.

In certain embodiments, the heavy chain of anti-CD19 antibody is at least 90% identical to SEQ ID NO: 54. In certain embodiments, the heavy chain of anti-CD19 antibody is at least 95% identical to SEQ ID NO: 54. In certain embodiments, the heavy chain of anti-CD19 antibody is at least 98% identical to SEQ ID NO: 54. In certain embodiments, the heavy chain of anti-CD19 antibody is identical to SEQ ID NO: 54.

In certain embodiments, the light chain of anti-CD19 antibody is at least 90% identical to SEQ ID NO: 55. In certain embodiments, the light chain of anti-CD19 antibody is at least 95% identical to SEQ ID NO: 55. In certain embodiments, the light chain of anti-CD19 antibody is at least 98% identical to SEQ ID NO: 55. In certain embodiments, the light chain of anti-CD19 antibody is identical to SEQ ID NO: 55.

### Fc Portion

In certain embodiments, the antibody variable domains of the antibodies prepared using the methods of the invention are optionally fused to an Fc portion. As used herein, the Fc portion encompasses domains derived from the heavy chain constant region of an immunoglobulin, preferably a human immunoglobulin, including a fragment, analog, variant, mutant or derivative of the constant region. The constant region of an immunoglobulin heavy chain is defined as a naturally-occurring or synthetically produced polypeptide homologous to at least a portion of the C-terminal region of the heavy chain, including the CH1, hinge, CH2, CH3, and, for some heavy chain classes, CH4 domains. The “hinge” region joins the CH1 domain to the CH2-CH3 region of an Fc portion. The constant region of the heavy chains of all mammalian immunoglobulins exhibit extensive amino acid sequence similarity.

In the present invention, the Fc portion typically includes at least a CH2 domain. For example, the Fc portion can include the entire immunoglobulin heavy chain constant region (CH1-hinge-CH2-CH3). Alternatively, the Fc portion can include all or a portion of the hinge region, the CH2 domain and the CH3 domain.

The constant region of an immunoglobulin is responsible for many important antibody effector functions, including those mediated by Fc receptor (FcR) binding and by complement binding. There are five major classes of the heavy chain constant region, classified as IgA, IgG, IgD, IgE, and IgM, each with characteristic effector functions designated by isotype.

IgG, for example, is separated into four γ isotypes: γ1, γ2, γ3, and γ4, also known as IgG1, IgG2, IgG3, and IgG4, respectively. IgG molecules can interact with multiple classes of cellular receptors including three classes of Fcγ receptors (FcγR) specific for the IgG class of antibody, namely FcγRI, FcγRII, and FcγRIII. The sequences important for the binding of IgG to the FcγR receptors have been reported to be in the CH2 and CH3 domains.

It is also often useful to alter the serum half-life of the antibody. The serum half-life of an antibody, as of an immunoglobulin fusion protein, is influenced by the ability of that antibody to bind to an Fc receptor (FcR) (Gillies et al., Cancer Research (1999) 59:2159-66). The CH2 and CH3 domains of IgG2 and IgG4 have undetectable or reduced binding affinity to Fc receptors compared to those of IgG1. Accordingly, the serum half-life of the featured antibody can be increased by using the CH2 and/or CH3 domain from IgG2 or IgG4 isotypes. Alternatively, the antibody can include a CH2 and/or CH3 domain from IgG1 or IgG3 with modification in one or more amino acids in these domains to reduce the binding affinity for Fc receptors (see, e.g., U.S. patent application Ser. No. 09/256,156, published as U.S. patent application publication 2003-0105294).

In certain embodiments, an Fc portion fused to an antibody variable region of the invention can contain CH2 and/or CH3 domains and a hinge region that are derived from different antibody isotypes. For example, the Fc portion can contain CH2 and/or CH3 domains of IgG2 or IgG4 and a hinge region of IgG1. Assembly of such hybrid Fc portions has been described in U.S. patent application publication 2003-0044423.

When fused to an antibody variable region of the invention, the Fc portion may contain one or more amino acid modifications that generally extend the serum half-life of an Fc fusion protein. Such amino acid modifications include mutations substantially decreasing or eliminating Fc receptor binding or complement fixing activity. For example, one type of such mutation removes the glycosylation site of the Fc portion of an immunoglobulin heavy chain. In IgG1, the glycosylation site is Asn297 (see, for example, U.S. patent application Ser. No. 10/310,719, published as U.S. patent application publication 2003-0166163).

### MCD and FSGS:

Minimal change disease (MCD) and primary focal segmental glomerulosclerosis (FSGS) are similar podocytopathies that are characterized by nephrotic syndrome, profound proteinuria, and near complete or complete foot processes effacement on biopsy. In many instances, these podocytopathies respond rapidly and completely to administration of corticosteroids, in which case they are defined as steroid sensitive.

Although the pathophysiology is not entirely certain, there is some evidence to suggest that circulating autoantibodies play a role in MCD. The symptoms of MCD include a sudden onset of nephrotic syndrome and profound edema.

The epidemiology of MCD is about 1 in 150,000 adults. There is an estimated population of 20,000 addressable patients in the United States. The uncontrolled disease results in complications of nephrotic syndrome and eventual kidney failure. Moreover, patients who respond to therapy can achieve long-term remissions. The conventional standard of care provides the use of steroids to induce and maintain remission. There is currently no FDA approved therapy for the treatment of MCD.

The available options for treatment (apart from steroids) include calcineurin inhibitors (CNIs), cyclophosphamide, and rituximab. The use of CNIs leads to nephrotoxicity and/or high relapse rate. The treatment with cyclophosphamide has a side effect of high toxicity. Rituximab also does not provide an efficacious treatment of MCD. Clin Kidney J. 2020 Nov. 21; 14(4):1042-1054. N Engl J Med. 2024 Aug. 1; 391(5):422-433.

### Methods of the Invention:

There is an unmet need for improved therapies for steroid-dependent and frequently relapsing MCD and FSGS, and there are currently no Food and Drug Administration-approved treatments specifically indicated for primary MCD or primary FSGS. Patients with MCD and FSGS suffer frequent disease flares and complications from long-term corticosteroid exposure. The invention provides a novel therapeutic approach for reducing inflammation and injury to kidney through depletion of CD19+ cells through compositions comprising anti-CD19 antibodies. The invention provides methods of treatment of glomerulonephropathy associated with MCD and FSGS. In certain beneficial aspects, the methods provided herein reduce proteinuria and maintain remission in MCD/FSGS patients.

A significant challenge in the treatment of patients with MCD is that a significant number of patients have become steroid dependent, which is the current standard of care for the treatment of MCD patients. These patients are unable to taper steroids without a relapse. In addition, this is a frequently relapsing disease.

In certain aspects, the invention provides a method of treating a subject having glomerulonephropathy, wherein the method comprises administration of a composition comprising a therapeutically effective amount of an anti-CD19 antibody. In certain embodiments, glomerulonephropathy is associated with the generation of autoantibodies. In certain embodiments, glomerulonephropathy is selected from the group consisting of minimal change disease (MCD), focal segmental glomerulosclerosis (FSGS), and lupus nephritis (LN). In certain embodiments, the invention provides that immunomodulation by anti-CD19 antibody and reduction in number of CD19+ B-cells.

In certain embodiments, the patients are corticosteroid-dependent or frequently relapsing. In certain embodiments, the subject has previously received corticosteroid therapy and/or any additional therapy for treatment of nephrotic syndromes. In certain embodiments, the additional therapies are selected from the group consisting of calcineurin inhibitors, mycophenolic acid analogues, azathioprine, and cyclophosphamide. In certain embodiments, the subject has received the corticosteroid therapy for at least 8 weeks before start of administration of the composition comprising a therapeutically effective amount of anti-CD19 antibody. In certain embodiments, the corticosteroid therapy is administration of prednisone. In certain embodiments, the prednisone is administered at a dose higher than 20 mg/day, the subject is tapered off to a dose of 20 mg/day of prednisone. In certain embodiments, tapering is done from at least eight weeks to about 2 weeks before starting administration of the composition. In certain embodiments, prednisone is administered at a dose of less than about 20 mg/day for at least two weeks before starting administration of the composition.

In certain embodiments, the anti-CD19 antibody of the invention could be any anti-CD19 antibody provided herein. In certain embodiments, wherein the anti-CD19 antibody comprises: a heavy chain variable region at least 95% identical to SEQ ID NO: 17 and/or a light chain variable region at least 95% identical to SEQ ID NO: 29. In certain embodiments, the anti-CD19 antibody is budoprutug.

In certain embodiments, the composition comprising a therapeutically effective amount of anti-CD19 antibody is administered parenterally. In certain embodiments, the composition comprising a therapeutically effective amount of anti-CD19 antibody is administered via intravenous (IV) infusion.

In certain embodiments, the composition comprising a therapeutic effective amount of anti-CD19 antibody is administered at days 1 and 15 at the start of the treatment. In certain embodiments, the composition comprises about 100 mg to about 500 mg of the anti-CD19 antibody. In certain embodiments, the composition comprises about 100 mg to about 400 mg of the anti-CD19 antibody. In certain embodiments, composition comprises about 100 mg to about 300 mg of the anti-CD19 antibody. In certain embodiments, composition comprises about 100 mg to about 200 mg of the anti-CD19 antibody. In certain preferred embodiments, composition comprises about 100 mg of the anti-CD19 antibody. In certain preferred embodiments, composition comprises about 200 mg of the anti-CD19 antibody.

In certain embodiments, the method further comprises administration of an additional cycle to the subject, wherein the additional cycle comprises administration of a first additional dose of the composition and a second additional dose of the composition. In certain embodiments, the concentration of B cells is measured to decide if the additional cycle is administered. A high B cell count indicates that the administration of the composition of the invention did not have the desired effect. In certain embodiments, the additional cycle is administered if B cell count in the subject is greater than 10 cells/μL.

In certain embodiments, the first additional dose of the additional cycle is administered between day 113 and day 155 after first administration of the composition to the subject. In certain embodiments, the first additional dose of the additional cycle is administered between day 113 and day 141 after first administration of the composition to the subject. In certain embodiments, the second additional dose is administered 14 days after administration of the first additional dose.

In certain embodiments, the first additional dose of the additional cycle is administered between day 169 and day 183 after first administration of the composition to the subject. In certain embodiments, the first additional dose and the second additional dose are administered if the subject has not previously received the first additional dose and the second additional dose between days 113 and 115 after first administration of the composition to the subject. In certain embodiments, the first additional dose and the second additional dose comprise about 100 mg of the anti-CD19 antibody. In certain embodiments, the first additional dose and the second additional dose comprise about 200 mg of the anti-CD19 antibody.

In a Phase Ib clinical study, a 57-year old patient with frequently relapsing MCD was enrolled. The patient relapsed on tacrolimus. The remission occurred after steroid course and relapsed within 2 months and steroids were reinitiated.

The subject received budoprutug, 2 induction doses and 2 maintenance doses. B cells were effectively depleted after the administration of the doses of budoprutug. The steroids were discontinued and the patient remained in remission 18 months after the last infusion. There were no safety related outcomes.

A protocol for the Phase 2 Study of budoprutug in Adult Subjects with Steroid-Sensitive Primary Minimal Change Disease or Primary Focal Segmental Glomerulosclerosis

| Objectives | Endpoints |
|---|---|
| Primary Efficacy Objective: | Primary Efficacy Endpoint: |
| To assess the efficacy of | The proportion of subjects in remission |
| VB119 on maintenance of | (UPCR <0.5 g/g) at Day 274 (EOT). |
| remission defined as <0.5 g/g | |
| UPCR in subjects with MCD | |
| or FSGS who were | |
| corticosteroid dependent or | |
| frequently relapsing. | |
| Primary Safety Objective: | Primary Safety Endpoints: |
| To evaluate the safety | Incidence of SAEs from the time of ICF |
| and tolerability of | signature to Day 420 (EOS); |
| VB119. | Incidence of TEAEs from the first dose |
| | of VB119 to Day 420 (EOS); |
| | Change from Day 1 in safety assessments |
| | (including clinical laboratory assessments, |
| | vital signs, electrocardiograms, and other |
| | assessments); and |
| | Incidence of AEs of special interest from |
| | the first dose of VB119 to Day 337 and Day |
| | 420 (EOS). |
| Secondary Efficacy Objectives: | Secondary Efficacy Endpoints: |
| To evaluate following: | Change in UPCR from Day 1 to Day 113, to |
| Change in proteinuria; | Day 337, and at multiple time points from |
| Proportion of subjects | Day 1 to Day 337; |
| with proteinuria | Proportion of subjects with UPCR <0.5 g/g |
| remaining at <0.5 g/g | and □0.3 g/g at multiple time points |
| and <0.3 g/g; | from Day 1 to Day 113 and to Day 337; |
| Change in renal | Change in eGFR from Screening to Day 113, |
| function; | to Day 274 (EOT), to Day 337, and at |
| Time to disease recurrence; and | multiple time points; |
| PK profile of VB119. | Change in eGFR slope from pre-treatment |
| | retrospective data (up to 2 years prior |
| | to Screening) to Day 113, to Day 274 (EOT), |
| | to Day 337, and at multiple time points; |
| | Proportion of subjects reaching a □40% |
| | decrease in eGFR from Day 1 to Day 113, to |
| | Day 274 (EOT), and to Day 337; |
| | Proportion of subjects that were recurrence- |
| | free (UPCR <3.5 g/g at multiple time points |
| | from Day 1 to Day 337); and |
| | PK profile of VB119 including but not limited |
| | to area under the time-concentration curve, |
| | time to maximum observed concentration, |
| | terminal half-life, apparent clearance, and |
| | volume of distribution. |
| Secondary Safety Objective: | Secondary Safety Endpoint: |
| To evaluate the | The proportion of subjects who develop ADAs |
| development of ADAs | (neutralizing and non-neutralizing) at any |
| to VB119. | time after study drug administration. |

ADA = anti-drug antibody; AE = adverse event; eGFR = estimated glomerular filtration rate; EOS = End of Study; EOT = End of Treatment; FSGS = focal segmental glomerulosclerosis; ICF = informed consent form; MCD = minimal change disease; PK = pharmacokinetic; SAE = serious adverse event; TEAE = treatment-emergent adverse event; UPCR = urine protein/creatinine ratio.

### Methodology:

This was a Phase 2, multi-center, proof-of-concept study to evaluate the safety and efficacy of VB119 on the maintenance of remission and duration of response in adults with primary MCD or primary FSGS who previously responded to steroid therapy. To be eligible for this study, subjects had to be in remission (urine protein/creatinine ratio [UPCR]□0.5 g/g) and meet the following criteria:
Were corticosteroid-dependent or frequently relapsing; and
Received corticosteroids and/or second-line therapies (eg, calcineurin inhibitor, mycophenolic acid analogues, azathioprine, or cyclophosphamide) to treat nephrotic syndrome at the time of Screening.

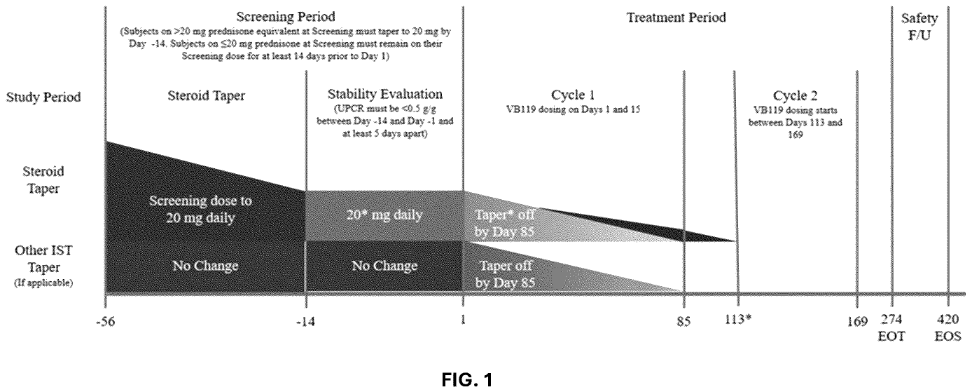

The study was designed to include up to 8-week Screening Period, a 9-month Treatment Period, and a 6-month Safety Follow-Up Period, as shown in the Study Schema (FIG. 1); however, the study was terminated before the Safety Follow-Up Period started. Early termination was not due to any safety concerns.

FIG. 1 provides the schematic of the design of the study.

### Screening Period: Up to 8 Weeks (Day −56 to Day −1)

Approximately 20 subjects were to be screened for this study with the goal of enrolling approximately 15 subjects, with at least 5 having FSGS.

Subjects who signed informed consent entered the Screening Period which included a Steroid Taper period from Day −56 to Day −14 and a Stability Evaluation period from Day −14 to Day −1. Subjects who entered the Screening Period on □20 mg prednisone equivalent had to taper to 20 mg by Day −14. Subjects on <20 mg prednisone equivalent at Screening were to begin Screening at Day −14. All subjects were to remain on a stable steroid dose <20 mg daily. A kidney biopsy confirming diagnosis for MCD or FSGS was performed prior to the subject being enrolled in the Treatment Period.

Treatment Period: 9 Months from the First Dose of VB119 (Day 1 to Day 274)

During the Treatment Period, eligible subjects were to receive a minimum of 2 intravenous doses of VB119: 100 mg or 200 mg (after Data Review Committee review) on Day 1 and 100 mg or 200 mg on Day 15 (200 mg or 400 mg cumulative dose). All prednisone was to be tapered off by Day 85 or Day 113. All non-prednisone immunosuppressive therapy (IST) was to be tapered off by Day 85 of the Treatment Period. Subjects were to receive 2 additional VB119 doses of 200 mg during the study when they met 1 of the following 2 criteria:
Between Day 113 (as measured on Day 85) and Day 155 (as measured on Day 113) of the Treatment Period if CD19+ B cell repopulation reached ≥10 cells/μL; or
   If the first additional dose was administered on Day 113, then a second additional dose was to be administered 14 days later on Day 127; or
   If the first additional dose was administered on Day 141, then a second additional dose was to be administered 14 days later on Day 155.
At Day 169 of the Treatment Period irrespective of CD19+ B cell count, unless they had previously received additional doses between Day 113 and Day 155 based on B cell repopulation.
   If the first additional dose was administered on Day 169, then a second additional dose was to be administered 14 days later on Day 183.

Any subject that had a relapse of proteinuria during the Treatment Period on or after Day 113, defined as proteinuria UPCR >3.5 g/g, was to be considered a treatment failure and was not to receive additional VB119 doses. Subjects experiencing worsening proteinuria prior to Day 113 in the Treatment Period were to receive rescue medications (steroids or other IST) to control flare per Protocol if complete withdrawal of steroids was to be completed by Day 113. If rescue therapy was required on or after Day 113, the Investigator was to discontinue VB119 treatment. Subjects who received rescue medication (steroids or other IST) continued to be monitored per study requirements as indicated in the Schedule of Procedures.

### Safety Follow-Up: 6 Months Following the Treatment Period (Day 275 to Day 420)

Following the end of the Treatment Period, subjects were to be followed for safety for 6 months, during which time subjects would be assessed for safety, renal function, B cell repopulation, and quality of life.
Due to early study termination, the Safety Follow-Up period did not occur.

### Duration of Treatment:

The total study duration for each individual subject was to be up to 17 months, including the Screening Period (up to 2 months), Treatment Period (9 months), and Safety Follow-Up Period (up to 6 months).

### Number of Subjects:

Planned: Approximately 20 subjects

### Diagnosis and Main Criteria for Inclusion:

Key study inclusion criteria included, but were not limited to, men and women ≥18 years of age with a kidney biopsy-proven diagnosis of primary MCD or primary FSGS within the past 10 years; a history of steroid-sensitive MCD or FSGS (defined as having achieved complete remission of proteinuria [reduction of proteinuria to <0.5 g/g UPCR] after use of corticosteroids); proteinuria in the past 2 years; on prednisone (or IST) prior to Screening and expected to be tapered to a stable dose of <20 mg/day; and with disease considered in complete remission prior to Screening.

### Investigational Product Information: VB119 (100 mg and 200 mg) Statistical Methods:

Since there was only 1 subject enrolled at the time of early study termination, only subject data listings were generated, and no statistical analysis was performed.

### Summary of Results:

### Disposition and Demographics:

Screened: 3 subjects
Randomized: 1 subject
Completed: 0 subjects

A single subject was enrolled in the study and received the first dose of VB119 100 mg on Day 1. The last study visit was on Day 238 due to early study termination by the Sponsor (Listing 16.2.1.1).

Subject 110-002 was a white female between 50 and 60 years of age with a BMI of 26.5 kg/m² (Listing 16.2.4.1). The subject had a relevant ongoing medical history of glomerulonephritis minimal lesion, nephrotic syndrome, and proteinuria and a prior disease history of frequently relapsing steroid sensitive MCD (Listings 16.2.4.2 and 16.2.4.3).

Subject 110-002 received prednisone 7.5 mg once daily from Day −42 until Day 85.
Extent of Exposure: The subject received 4 doses of VB119 (Table 1) and completed the Treatment Period before the study was terminated early by the Sponsor.
Efficacy Results: Due to early study termination and having data from only 1 subject, no formal efficacy analyses could be performed.

UPCR was measured from the first morning void spot urine sample, which was then normalized to creatinine to account for urine concentration. Estimated glomerular filtration rate (eGFR) was calculated from serum creatinine using the Chronic Kidney Disease Epidemiology Collaboration equation to estimate renal function through estimated clearance.

The primary efficacy parameter, UPCR, was measured from Screening through End of Treatment (EOT) (Day 238) and ranged from 47 mg/g to 101 mg/g (normal range [NR]: <200 mg/g) (Table 1). The UPCR results showed that proteinuria was well-controlled and remained in remission (<0.5 g/g) throughout the study.

TABLE 1
Primary Efficacy Parameter: UPCR - All Treated Subjects

| | | | |
|---|---|---|---|
| | | VB119 | |
| Subject | Study Visit | Dose | |
| Parameter | (Day) [1] | Received | Result |

110-002

| | | | |
|---|---|---|---|
| Protein creatinine | Screening Day A | — | 49 |
| ratio (mg/g) | (Day −42) | | |
| (Normal range: | Unscheduled Visit | — | 50 |
| □200 mg/g) | (Day −14) | | |
| | Screening Day B | — | 51 |
| | (Day −8) | | |
| | Day 1 (Day 1) [2] | 100 mg | NC |
| | Day 8 (Day 8) | — | 49 |
| | Day 15 (Day 15) | 100 mg | 101 |
| | Day 22 (Day 22) | — | 56 |
| | Day 29 (Day 29) | — | 55 |
| | Day 50 (Day 50) | — | 70 |
| | Day 71 (Day 71) | — | NC |
| | Day 85 (Day 85) | — | 50 |
| | Day 113 (Day 113) | — | 87 |
| | Unscheduled Visit | — | 51 |
| | (Day 127) | | |
| | Day 141 (Day 141) | — | NC |
| | Unscheduled Visit | — | 57 |
| | (Day 155) | | |
| | Day 169 (Day 175) | 200 mg | 47 |
| | Day 183 (Day 189) | 200 mg | 54 |
| | Day 197 (Day 204) | — | 55 |
| | Day 274 (EOT) | — | 54 |
| | (Day 238) | | |

[1]. Day = date - date of first dose (+1 if date ≥ date of first dose).
[2]. Indicates baseline as last available assessment before first dose of study drug.
EOT = End of Treatment; NC = not calculated; UPCR = urine protein creatinine ratio.

The secondary efficacy parameter, eGFR, was measured from Screening through EOT (Day 238) and ranged from 67 mL/min/1.73 m² to 94 mL/min/1.73 m² (NR: >53 mL/min/1.73 m²) throughout the study. The eGFR results showed that renal function was normal for the subject throughout the study.

No edema was reported by the subject.
Pharmacokinetic Results: There was no drug concentration data for Subject 110-002.
Safety Results: Subject 110-002 experienced 1 treatment-emergent adverse event (TEAE) of nodule (reported term: right axillary nodule) on Day 155, which resolved on Day 189. This TEAE was considered mild in severity and not related to study drug. No infusion-related reactions occurred.

The safety laboratory parameter serum creatinine was measured from Screening through EOT (Day 238) and ranged from 0.74 mg/dL to 0.98 mg/dL (NR: 0.49 mg/dL to 1.12 mg/dL) remaining within the NR throughout the study. Other liver and renal function parameters (alanine aminotransferase, albumin, alkaline phosphatase, aspartate aminotransferase, bilirubin, creatine kinase, gamma-glutamyl transferase, and urea nitrogen) also remained within the NRs.
No anti-drug antibodies (ADAs) were reported.

Conclusions: In this Phase 2 study, 1 subject was enrolled and received 4 doses of VB119.

There were no safety concerns related to the administration of VB119 including the absence of any critical laboratory value, with liver and renal function parameters including creatinine remaining within the NR. Subject 110-002 experienced only 1 TEAE (nodule) that was mild in severity and not related to the study drug. There were no infusion-related reactions or ADAs.

The table below provides the sequences for nucleic acids, proteins, and peptides discussed herein.

| SEQ ID | |
|---|---|
| NO: | Sequence |
| 1 | CAGGTGCAACTGCAGCAGCCTGGGGCTGAAGTGGTGAAGCCTGGGGC |
| | TTCAGTGAGACTGTCCTGCAAGACTTCTGGCTACACCTTCACCAGCAA |
| | CTGGATGCACTGGGTGAAGCAGAGGCCTGGACAAGGCCTTGAGTGGA |
| | TCGGAGAGATTGATCCTTCTGATAGTTATACTAACTACAATCAAAAGTT |
| | CAAGGGCAAGGCCAAGTTGACTGTAGACAAATCCTCCAGCACAGCCT |
| | ACATGGAAGTCAGCAGCCTGACATCTGAGGACTCTGCGGTCTATTACT |
| | GTGCAAGAGGTAGCAACCCTTACTACTATGCTATGGACTACTGGGGTCA |
| | AGGAACCTCAGTCACCGTCTCCTCA |
| 2 | CAGGTGCAACTGCAGCAGCCTGGGGCTGAAGTGGTGAAGCCTGGGGC |
| | TTCAGTGAGACTGTCCTGCGAGACTTCTGGCTACACCTTCACCAGCAA |
| | CTGGATGCACTGGGTGAAGCAGAGGCCTGACCAAGGACTTGAGTGGA |
| | TCGGAGAGATTGATCCTTCTGATAGTTATACTAACTACAATCAAAAGTT |
| | CAAGGGCAAGGCCGAATTGACTGTAGACAAATCCTCCAGCACAGCCTA |
| | CATGGAAGTCAGCGACCTGACATCTGAGGACTCTGCGGTCTATTACTGT |
| | GCAAGAGGTAGCAACCCTTACTACTATGCTATGGACTACTGGGGTCAA |
| | GGAACCTCAGTCACCGTCTCCTCA |
| 3 | CAGGTGCAACTGCAGCAGCCTGGGGCTGAAGTGGTGAAGCCTGGGGC |
| | TTCAGTGAGACTGTCCTGCAAGACTTCTGGCTACACCTTCACCAGCAA |
| | CTGGATGCACTGGGTGAAGCAGAGACCTGGACAAGGACTTGAGTGGA |
| | TCGGAGAGATTGATCCTTCTGATAGTTATACTAACTACAATCAAAAGTT |
| | CAAGGGCAAGGCCGAATTGACTGTAGACAAATCCTCCAGCACAGCCTA |
| | CATGGAAGTCAGCGACCTGACATCTGAGGACTCTGCGGTCTATTACTGT |
| | GCAAGAGGTAGCAACCCTTACTACTATGCTATGGACTACTGGGGTCAA |
| | GGAACCTCAGTCACCGTCTCCTCA |
| 4 | CAGGTGCAACTGGAGCAGCCTGGGGCTGAAGTGAAGAAGCCTGGGGC |
| | TTCAGTGAAGGTGTCCTGCAAGGCTTCTGGCTACACCTTCACCAGCAA |
| | CTGGATGCACTGGGTGAAGCAGAGGCCTGGACAAGGACTTGAGTGGA |
| | TCGGAGAGATTGATCCTTCTGATAGTTATACTAACTACAATCAAAAGTT |
| | CAAGGGCAAGGCCAAGTTGACTGTAGACAAATCCTCCAGCACAGCCT |
| | ACATGGAAGTCAGCGACCTGACAGCTGAGGACTCTGCGGTCTATTACT |
| | GTGCAAGAGGTAGCAACCCTTACTACTATGCTATGGACTACTGGGGTCA |
| | AGGAACCTCAGTCACCGTCTCCTCA |
| 5 | CAGGTGCAACTGGAGCAGCCTGGGGCTGAAGTGGTGAAGCCTGGGGC |
| | TTCAGTGAAGGTGTCCTGCAAGACTTCTGGCTACACCTTCACCAGCAA |
| | CTGGATGCACTGGGTGAAGCAGACGCCTGGAAAAGGACTTGAGTGGA |
| | TCGGAGAGATTGATCCTTCTGATAGTTATACTAACTACAATCAAAAGTT |
| | CGATGGCAAGGCCAAGTTGACTGTAGACAAATCCTCCAGCACAGCCTA |
| | CATGGAAGTCAGCGACCTGACAGCTGAGGACTCTGCGACCTATTACTG |
| | TGCAAGAGGTAGCAACCCTTACTACTATGCTATGGACTACTGGGGTCAA |
| | GGAACCTCAGTCACCGTCTCCTCA |
| 6 | CAGGTGCAACTGGAGCAGCCTGGGGCTGAAGTGAAGAAGCCTGGGGC |
| | TTCAGTGAAGGTGTCCTGCAAGGCTTCTGGCTACACCTTCACCAGCAA |
| | CTGGATGCACTGGGTGAGACAGGCACCTGGAAAAGGACTTGAGTGGA |
| | TCGGAGAGATTGATCCTTCTGATAGTTATACTAACTACAATCAAAAGTT |
| | CGATGGCAAGGCCAAGTTGACTGTAGACAAATCCTCCAGCACAGCCTA |
| | CATGGAAGTCAGCGACCTGACATCTGAGGACTCTGCGACCTATTACTG |
| | TGCAAGAGGTAGCAACCCTTACTACTATGCTATGGACTACTGGGGTCAA |
| | GGAACCTCAGTCACCGTCTCCTCA |
| 7 | CAGGTGCAACTGGAGCAGCCTGGGGCTGAAGTGGTGAAGCCTGGGGC |
| | TTCAGTGAAGGTGTCCTGCAAGACTTCTGGCTACACCTTCACCAGCAA |
| | CTGGATGCACTGGGTGAAGCAGAGGCCTGGACAAGGACTTGAGTGGA |
| | TCGGAGAGATTGATCCTTCTGATAGTTATACTAACTACAATCAAAAGTT |
| | CGATGGCAAGGCCAAGTTGACTGTAGACAAATCCTCCAGCACAGCCTA |
| | CATGGAAGTCAGCGACCTGACATCTGAGGACTCTGCGACCTATTACTG |
| | TGCAAGAGGTAGCAACCCTTACTACTATGCTATGGACTACTGGGGTCAA |
| | GGAACCTCAGTCACCGTCTCCTCA |
| 8 | CAAATTGTTCTCACCCAGTCTCCAGCAATCATGTCTGCATCTCCAGGGG |
| | AGAAGGTCACCATGACCTGCAGTGCCAGCTCAGGTGTCAACTACATGC |
| | ACTGGTACCAGCAGAAGCCAGGCACCTCCCCCAAAAGATGGATTTATG |
| | ACACATCCAAACTGGCTTCTGGAGTCCCTGCTCGCTTCAGTGGCAGTG |
| | GGTCTGGGACCTCTTATTCTCTCACAATCAGCAGCATGGAGGCTGAAG |
| | ATGCTGCCACTTATTACTGCCATCAGCGAGGTAGTTACACGTTCGGAGG |
| | GGGGACCAAGCTGGAAATAAA |
| 9 | CAAATTGCTCTCACCCAGGAGCCAGCAATCATGTCTGCATCTCCAGGG |
| | GAGAAGGTCACCATGACCTGCAGTGCCAGCTCAGGTGTCAACTACATG |
| | CACTGGTATCAGCAGAAGCCAGGCACCTCCCCCAAAAGATGGATTTAT |
| | GACACATCCAAACTGGATTCTGGAGTCCCTGCTCGCTTCAGTGGCAGT |
| | GGGTCTGGGACCTCTTATTCTCTCACAATCAGCAGCATGGAGGCTGAA |
| | GATGCTGCCACTTATTACTGCCATCAGCGAGGTAGTTACACGTTCGGAG |
| | GGGGGACCAAGCTGGAAATAAA |
| 10 | GACATTGTTCTCACCCAGTCTCCAGCAACTTTGTCTGCATCTCCAGGGG |
| | AGAAGGTCACCATGACCTGTAGTGCCAGCTCAGGTGTCAACTACATGC |
| | ACTGGTATCAGCAGAAGCCAGGCACCTCCCCCAAAAGATGGATTTATG |
| | ACACATCCAAACTGGATTCTGGAGTCCCTGCTCGCTTCAGTGGCAGTG |
| | GGTCTGGGACCTCTTATTCTCTCACAATCAGCAGCATGGAGGCTGAAG |
| | ATGCTGCCACTTATTACTGCCATCAGCGAGGTAGTTACACGTTCGGAGG |
| | GGGGACCAAGCTGGAAATAAA |
| 11 | CAAATTGTTCTCACCCAGTCTCCAGCAACTTTGTCTGCATCTCCAGGGG |
| | AGAAGGCTACCATGACCTGCAGTGCCAGCTCAGGTGCTAACTACATGC |
| | ACTGGTACCAGCAGAAGCCAGGCACCTCCCCCAAAAGATGGATTTATG |
| | ACACATCCAAACTGGCTTCTGGAGTCCCTGCTCGCTTCAGTGGCAGTG |
| | GGTCTGGGACCTCTTATTCTCTCACAATCGAGAGCATGGAGGCTGAAG |
| | ATGCTGCCACTTATTACTGCCATCAGCGAGGTAGTTACACGTTCGGAGG |
| | GGGGACCAAGCTGGAAATAAA |
| 12 | CAAATTGTTCTCACCCAGTCTCCAGCAACTTTGTCTGCATCTCCAGGGG |
| | AGAAGGCTACATGACCTGTAGTGCCAGCTCAGGTGTCAACTACATGCA |
| | CTGGTACCAGCAGAAGCCAGGCACCTCCCCCAAAAGATGGATTTATGA |
| | CACAGACAAAACGGCTTCTGGAGTCCCTGCTCGCTTCAGTGGCAGTGG |
| | GTCTGGGACCTCTTATTCTCTCACAATCAGCAGCATGGAGGCTGAAGAT |
| | GCTGCCACTTATTACTGCCATCAGCGAGGTAGTTACACGTTCGGAGGG |
| | GGGACCAAGCTGGAAATAAA |
| 13 | QVQLQQPGAEVVKPGASVRLSCKTSGYTFTSNWMHWVKQRPGQGLEWI |
| | GEIDPSDSYTNYNQKFKGKAKLTVDKSSSTAYMEVSSLTSEDSAVYYCAR |
| | GSNPYYYAMDYWGQGTSVTVSS |
| 14 | QVQLQQPGAEVVKPGASVRLSCETSGYTFTSNWMHWVKQRPDQGLEWI |
| | GEIDPSDSYTNYNQKFKGKAELTVDKSSSTAYMEVSDLTSEDSAVYYCAR |
| | GSNPYYYAMDYWGQGTSVTVSS |
| 15 | QVQLQQPGAEVVKPGASVRLSCKTSGYTFTSNWMHWVKQRPGQGLEWI |
| | GEIDPSDSYTNYNQKFKGKAELTVDKSSSTAYMEVSDLTSEDSAVYYCAR |
| | GSNPYYYAMDYWGQGTSVTVSS |
| 16 | QVQLEQPGAEVKKPGASVKVSCKASGYTFTSNWMHWVKQRPGQGLEW |
| | IGEIDPSDSYTNYNQKFKGKAKLTVDKSSSTAYMEVSDLTAEDSAVYYCA |
| | RGSNPYYYAMDYWGQGTSVTVSS |
| 17 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSS |
| 18 | QVQLEQPGAEVKKPGASVKVSCKASGYTFTSNWMHWVRQAPGKGLEW |
| | IGEIDPSDSYTNYNQKFDGKAKLTVDKSSSTAYMEVSDLTSEDSATYYCA |
| | RGSNPYYYAMDYWGQGTSVTVSS |
| 19 | QVQLEQPGAEVVKPGASVKVSCKTSGYTFTSNWMHWVKQRPGQGLEWI |
| | GEIDPSDSYTNYNQKFDGKAKLTVDKSSSTAYMEVSDLTSEDSATYYCAR |
| | GSNPYYYAMDYWGQGTSVTVSS |
| 20 | QVQLQQPGAEVKKPGASVRLSCETSGYTFTSNWMHWVKQRPDQGLEWI |
| | GEIDPSDSYTNYNQKFDGKAELTVDKSSSTAYMEVSDLTSEDSAVYYCAR |
| | GSNPYYYAMDYWGQGTSVTVSS |
| 21 | QVQLEQPGAEVKKPGASVKVSCKASGYTFTSNWMHWVKQTPGKGLEW |
| | IGEIDPSDSYTNYNQKFDGKAKLTVDKSSSTAYMEVSDLTAEDSATYYCA |
| | RGSNPYYYAMDYWGQGTSVTVSS |
| 22 | QVQLXQPGAEVXKPGASVXXSCXXSGYTF |
| 23 | WVXQXPXXGLEWIG |
| 24 | YNQKFXGKAXLTVDKSSSTAYMEVSXLTXEDSAXYYCA |
| 25 | QIVLTQSPAIMSASPGEKVTMTCSASSGVNYMHWYQQKPGTSPKRWIYD |
| | TSKLASGVPARFSGSGSGTSYSLTISSMEAEDAATYYCHQRGSYTFGGGT |
| | KLEIK |
| 26 | QIALTQEPAIMSASPGEKVTMTCSASSGVNYMHWYQQKPGTSPKRWIYD |
| | TSKLDSGVPARFSGSGSGTSYSLTISSMEAEDAATYYCHQRGSYTFGGGT |
| | KLEIK |
| 27 | DIVLTQSPATLSASPGEKVTMTCSASSGVNYMHWYQQKPGTSPKRWIYDT |
| | SKLDSGVPARFSGSGSGTSYSLTISSMEAEDAATYYCHQRGSYTFGGGTK |
| | LEIK |
| 28 | QIVLTQSPATLSASPGEKATMTCSASSGANYMHWYQQKPGTSPKRWIYDT |
| | SKLASGVPARFSGSGSGTSYSLTIESMEAEDAATYYCHQRGSYTFGGGTK |
| | LEIK |
| 29 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIK |
| 30 | QIALTQEPAIMSASPGEKATMTCSASSGVNYMHWYQQKPGTSPKRWIYD |
| | TSKLDSGVPARFSGSGSGTSYSLTIESMEAEDAATYYCHQRGSYTFGGGT |
| | KLEIK |
| 31 | QIVLTQSPATLSASPGEKATMTCSASSGANYMHWYQQKPGTSPKRWIYDT |
| | DKTASGVPARFSGSGSGTSYSLTIESMEAEDAATYYCHQRGSYTFGGGTK |
| | LEIK |
| 32 | XIXLTQXPAXXSASPGEKXTMTC |
| 33 | DTXKXX |
| 34 | QVQLQQPGAEVVKPGASVRLSCKTSGYTFTSNWMHWVKQRPGQGLEWI |
| | GEIDPSDSYTNYNQKFKGKAKLTVDKSSSTAYMEVSSLTSEDSAVYYCAR |
| | GSNPYYYAMDYWGQGTSVTVSS |
| 35 | QIVLTQSPAIMSASPGEKVTMTCSASSGVNYMHWYQQKPGTSPKRWIYD |
| | TSKLASGVPARFSGSGSGTSYSLTISSMEAEDAATYYCHQRGSYTFGGGT |
| | KLEIK |
| 36 | VRLSCKTSG |
| 37 | WVKQRPGQG |
| 38 | YNQKFKGKA |
| 39 | FKGKAKLTV |
| 40 | YMEVSSLTS |
| 41 | VYYCARGSN |
| 42 | IVLTQSPAI |
| 43 | VLTQSPAIM |
| 44 | VTMTCSASS |
| 45 | VNYMHWYQQ |
| 46 | WIYDTSKLA |
| 47 | IYDTSKLAS |
| 48 | CGTAAGTGGATCC |
| 49 | GCTAGCTCCAGC |
| 50 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLS |
| | PGK |
| 51 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLS |
| | PGK |
| 52 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |
| 53 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |
| 54 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLS |
| | PGK |
| 55 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of treating a subject having glomerulonephropathy, wherein the method comprises administration of a composition comprising a therapeutically effective amount of an anti-CD19 antibody, wherein the anti-CD19 antibody comprises: a heavy chain variable region at least 95% identical to SEQ ID NO: 17 and/or a light chain variable region at least 95% identical to SEQ ID NO: 29.

2. The method of claim 1, wherein the anti-CD19 antibody comprises a heavy chain variable region comprising SEQ ID NO: 17 and/or a light chain variable region comprising SEQ ID NO: 29, and preferably the anti-CD19 antibody is budoprutug.

3. The method of claim 2, wherein the composition is administered parenterally.

4. The method of claim 3, wherein the composition is administered via an intravenous (IV) infusion.

5. (canceled)

6. (canceled)

7. The method of claim 1, wherein the subject is corticosteroid-dependent or frequently relapsing.

8. The method of claim 7, wherein the subject has previously received corticosteroid therapy and/or any additional therapy for treatment of nephrotic syndromes.

9. The method of claim 8, wherein the additional therapies are selected from the group consisting of calcineurin inhibitors, mycophenolic acid analogues, azathioprine, and cyclophosphamide.

10. The method of claim 10, wherein the composition is administered at days 1 and 15 at the start of the treatment.

11. The method of claim 10, wherein the composition comprises about 100 mg to about 500 mg of the anti-CD19 antibody.

12. The method of claim 10, wherein the composition comprises about 100 mg to about 400 mg of the anti-CD19 antibody.

13. The method of claim 10, wherein the composition comprises about 100 mg to about 300 mg of the anti-CD19 antibody.

14. The method of claim 10, wherein the composition comprises about 100 mg of the anti-CD19 antibody.

15. The method of claim 10, wherein the composition comprises about 200 mg of the anti-CD19 antibody.

16. The method of claim 8, wherein the subject has received the corticosteroid therapy for at least 8 weeks before start of administration of the composition.

17. The method of claim 16, wherein the corticosteroid therapy is administration of prednisone.

18. The method of claim 17, wherein if the prednisone is administered at a dose higher than 20 mg/day, the subject is tapered off to a dose of 20 mg/day of prednisone.

19. The method of claim 18, wherein tapering is done from at least eight weeks to about 2 weeks before starting administration of the composition.

20. The method of claim 18, wherein prednisone is administered at a dose of less than about 20 mg/day for at least two weeks before starting administration of the composition.

21. (canceled)

22. (canceled)

23. The method of claim 20, wherein the method further comprises administration of an additional cycle to the subject, wherein the additional cycle comprises administration of a first additional dose of the composition and a second additional dose of the composition.

24. The method of claim 23, wherein the additional cycle is administered if B cell count in the subject is greater than 10 cells/μL.

25. (canceled)

26. (canceled)

27. (canceled)

28. (canceled)

29. (canceled)

30. (canceled)


SEQUENCE LISTING (55 sequences)

SEQ ID NO: 1 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagaggcctggacaaggccttgagtggatcggagagattgatccttctgatagttatactaactacaatcaaaagttcaagggcaaggccaagttgactgtagacaaatcctccagcacagcctacatggaagtcagcagcctgacatctgaggactctgcggtctattactgtgcaagaggtagcaacccttactactatgctatggactactggggtcaaggaacctcagtcaccgtctcctca

SEQ ID NO: 2 - DNA, 360 bp, synthetic construct
  source 1..360: mol_type = other DNA
caggtgcaactgcagcagcctggggctgaagtggtgaagcctggggcttcagtgagactgtcctgcgagacttctggctacaccttcaccagcaactggatgcactgggtgaagcagaggcctgaccaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 3 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagagacctggacaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 4 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgaagcagaggcctggacaaggacttgagtggatcggagagattgatccttctgatagttatactaactacaatcaaaagttcaagggcaaggccaagttgactgtagacaaatcctccagcacagcctacatggaagtcagcgacctgacagctgaggactctgcggtctattactgtgcaagaggtagcaacccttactactatgctatggactactggggtcaaggaacctcagtcaccgtctcctca

SEQ ID NO: 5 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagacgcctggaaaaggacttgagtggatcggagagattgatccttctgatagttatactaactacaatcaaaagttcgatggcaaggccaagttgactgtagacaaatcctccagcacagcctacatggaagtcagcgacctgacagctgaggactctgcgacctattactgtgcaagaggtagcaacccttactactatgctatggactactggggtcaaggaacctcagtcaccgtctcctca

SEQ ID NO: 6 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgagacaggcacctggaaaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 7 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagaggcctggacaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 8 - DNA, 311 bp, synthetic construct
  source 1..311: mol_type = other DNA
caaattgttctcacccagtctccagcaatcatgtctgcatctccaggggagaaggtcaccatgacctgcagtgccagctcaggtgtcaactacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 9 - DNA, 311 bp, synthetic construct
  source 1..311: mol_type = other DNA
caaattgctctcacccaggagccagcaatcatgtctgcatctccaggggagaaggtcaccatgacctgcagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 10 - DNA, 311 bp, synthetic construct
  source 1..311: mol_type = other DNA
gacattgttctcacccagtctccagcaactttgtctgcatctccaggggagaaggtcaccatgacctgtagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 11 - DNA, 311 bp, synthetic construct
  source 1..311: mol_type = other DNA
caaattgttctcacccagtctccagcaactttgtctgcatctccaggggagaaggctaccatgacctgcagtgccagctcaggtgctaactacatgcactggtaccagcagaagccaggcacctcccccaaaagatggatttatgacacatccaaactggcttctggagtccctgctcgcttcagtggcagtgggtctgggacctcttattctctcacaatcgagagcatggaggctgaagatgctgccacttattactgccatcagcgaggtagttacacgttcggaggggggaccaagctggaaataaa

SEQ ID NO: 12 - DNA, 310 bp, synthetic construct
  source 1..310: mol_type = other DNA
caaattgttctcacccagtctccagcaactttgtctgcatctccaggggagaaggctacatgacctgtagtgccagctcaggtgtcaactacatgcactggtaccagcagaagccaggcacctcccccaaaagatggatttatgacacagacaaaacggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 13 - protein, 120 aa, synthetic construct
QVQLQQPGAEVVKPGASVRLSCKTSGYTFTSNWMHWVKQRPGQGLEWIGEIDPSDSYTNYNQKFKGKAKLTVDKSSSTAYMEVSSLTSEDSAVYYCARGSNPYYYAMDYWGQGTSVTVSS

SEQ ID NO: 14 - protein, 120 aa, synthetic construct
QVQLQQPGAEVVKPGASVRLSCETSGYTFTSNWMHWVKQRPDQGLEWIGEIDPSDSYTNYNQKFKGKAELTVDKSSSTAYMEVSDLTSEDSAVYYCARGSNPYYYAMDYWGQGTSVTVSS

SEQ ID NO: 15 - protein, 120 aa, synthetic construct
QVQLQQPGAEVVKPGASVRLSCKTSGYTFTSNWMHWVKQRPGQGLEWIGEIDPSDSYTNYNQKFKGKAELTVDKSSSTAYMEVSDLTSEDSAVYYCARGSNPYYYAMDYWGQGTSVTVSS

SEQ ID NO: 16 - protein, 120 aa, synthetic construct
QVQLEQPGAEVKKPGASVKVSCKASGYTFTSNWMHWVKQRPGQGLEWIGEIDPSDSYTNYNQKFKGKAKLTVDKSSSTAYMEVSDLTAEDSAVYYCARGSNPYYYAMDYWGQGTSVTVSS

SEQ ID NO: 17 - protein, 120 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 18 - protein, 120 aa, synthetic construct
QVQLEQPGAEVKKPGASVKVSCKASGYTFTSNWMHWVRQAPGKGLEWIGEIDPSDSYTNYNQKFDGKAKLTVDKSSSTAYMEVSDLTSEDSATYYCARGSNPYYYAMDYWGQGTSVTVSS

SEQ ID NO: 19 - protein, 120 aa, synthetic construct
QVQLEQPGAEVVKPGASVKVSCKTSGYTFTSNWMHWVKQRPGQGLEWIGEIDPSDSYTNYNQKFDGKAKLTVDKSSSTAYMEVSDLTSEDSATYYCARGSNPYYYAMDYWGQGTSVTVSS

SEQ ID NO: 20 - protein, 120 aa, synthetic construct
QVQLQQPGAEVKKPGASVRLSCETSGYTFTSNWMHWVKQRPDQGLEWIGEIDPSDSYTNYNQKFDGKAELTVDKSSSTAYMEVSDLTSEDSAVYYCARGSNPYYYAMDYWGQGTSVTVSS

SEQ ID NO: 21 - protein, 120 aa, synthetic construct
QVQLEQPGAEVKKPGASVKVSCKASGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 22 - protein, 29 aa, synthetic construct
QVQLXQPGAEVXKPGASVXXSCXXSGYTF

SEQ ID NO: 23 - protein, 14 aa, synthetic construct
WVXQXPXXGLEWIG

SEQ ID NO: 24 - protein, 38 aa, synthetic construct
YNQKFXGKAXLTVDKSSSTAYMEVSXLTXEDSAXYYCA

SEQ ID NO: 25 - protein, 104 aa, synthetic construct
QIVLTQSPAIMSASPGEKVTMTCSASSGVNYMHWYQQKPGTSPKRWIYDTSKLASGVPARFSGSGSGTSYSLTISSMEAEDAATYYCHQRGSYTFGGGTKLEIK

SEQ ID NO: 26 - protein, 104 aa, synthetic construct
QIALTQEPAIMSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 27 - protein, 104 aa, synthetic construct
DIVLTQSPATLSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 28 - protein, 104 aa, synthetic construct
QIVLTQSPATLSASPGEKATMTCSASSGANYMHWYQQKPGTSPKRWIYDTSKLASGVPARFSGSGSGTSYSLTIESMEAEDAATYYCHQRGSYTFGGGTKLEIK

SEQ ID NO: 29 - protein, 104 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIK

SEQ ID NO: 30 - protein, 104 aa, synthetic construct
QIALTQEPAIMSASPGEKATMTCSASSGVNYMHWYQQKPGTSPKRWIYDTSKLDSGVPARFSGSGSGTSYSLTIESMEAEDAATYYCHQRGSYTFGGGTKLEIK

SEQ ID NO: 31 - protein, 104 aa, synthetic construct
QIVLTQSPATLSASPGEKATMTCSASSGANYMHWYQQKPGTSPKRWIYDTDKTASGVPARFSGSGSGTSYSLTIESMEAEDAATYYCHQRGSYTFGGGTKLEIK

SEQ ID NO: 32 - protein, 23 aa, synthetic construct
XIXLTQXPAXXSASPGEKXTMTC

SEQ ID NO: 33 - skipped

SEQ ID NO: 34 - protein, 120 aa, synthetic construct; identical to SEQ ID NO: 13

SEQ ID NO: 35 - protein, 104 aa, synthetic construct; identical to SEQ ID NO: 25

SEQ ID NO: 36 - protein, 9 aa, synthetic construct
VRLSCKTSG

SEQ ID NO: 37 - protein, 9 aa, synthetic construct
WVKQRPGQG

SEQ ID NO: 38 - protein, 9 aa, synthetic construct
YNQKFKGKA

SEQ ID NO: 39 - protein, 9 aa, synthetic construct
FKGKAKLTV

SEQ ID NO: 40 - protein, 9 aa, synthetic construct
YMEVSSLTS

SEQ ID NO: 41 - protein, 9 aa, synthetic construct
VYYCARGSN

SEQ ID NO: 42 - protein, 9 aa, synthetic construct
IVLTQSPAI

SEQ ID NO: 43 - protein, 9 aa, synthetic construct
VLTQSPAIM

SEQ ID NO: 44 - protein, 9 aa, synthetic construct
VTMTCSASS

SEQ ID NO: 45 - protein, 9 aa, synthetic construct
VNYMHWYQQ

SEQ ID NO: 46 - protein, 9 aa, synthetic construct
WIYDTSKLA

SEQ ID NO: 47 - protein, 9 aa, synthetic construct
IYDTSKLAS

SEQ ID NO: 48 - protein, 13 aa, synthetic construct
CGTAAGTGGATCC

SEQ ID NO: 49 - protein, 12 aa, synthetic construct
GCTAGCTCCAGC

SEQ ID NO: 50 - protein, 450 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 450 aa, synthetic construct; identical to SEQ ID NO: 50

SEQ ID NO: 52 - protein, 211 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 211 aa, synthetic construct; identical to SEQ ID NO: 52

SEQ ID NO: 54 - protein, 450 aa, synthetic construct; identical to SEQ ID NO: 50

SEQ ID NO: 55 - protein, 211 aa, synthetic construct; identical to SEQ ID NO: 52